(19) United States
(12) Patent Application Publication
(10) Pub. No.: US 2023/0340391 A1
(43) Pub. Date: Oct. 26, 2023

(54) SUBSTANCE INTRODUCTION DEVICE AND SUBSTANCE INTRODUCTION METHOD

(71) Applicant: TERUMO KABUSHIKI KAISHA, Tokyo (JP)

(72) Inventor: Youichi HIGUCHI, Kanagawa (JP)

(21) Appl. No.: 18/216,466

(22) Filed: Jun. 29, 2023

Related U.S. Application Data
(63) Continuation of application No. PCT/JP2022/008430, filed on Feb. 28, 2022.

(30) Foreign Application Priority Data
Mar. 8, 2021 (JP) 2021-036762

(51) Int. Cl. C12M 1/42

(52) U.S. Cl. CPC C12M 35/04; C12M 35/02

(57) ABSTRACT

A substance introduction device includes at least one turbulence grid that generates turbulence in a fluid containing a cell and a substance to be introduced into the cell, in which at least a part of the at least one turbulence grid is formed of a conductive material that applies charge to the cell in the fluid. A substance introduction method includes a stress/charge applying step of applying, while generating turbulence by flowing a fluid containing a cell and a substance to be introduced into the cell through at least one turbulence grid, charge to the cell in the fluid by a conductive material forming at least a part of the at least one turbulence grid.

### CROSS-REFERENCE TO RELATED APPLICATIONS

The present disclosure is a continuation of and claims benefit to PCT/JP2022/008430 filed on Feb. 28, 2022, entitled “SUBSTANCE INTRODUCTION DEVICE AND SUBSTANCE INTRODUCTION METHOD” which claims priority to Japanese Patent Application No. 2021-036762 filed on Mar. 8, 2021. The entire disclosure of the applications listed above are hereby incorporated herein by reference, in their entireties, for all that they teach and for all purposes.

### BACKGROUND

The present disclosure relates to a substance introduction device and a substance introduction method.

One of methods of introducing a substance such as a gene into a cell of an animal or a plant is a physical introduction method. Examples of the physical introduction method include charge application (electroporation), stress application (perforation method by shear stress) and the like, as described, for example, in Japanese Patent Publication No. 2018-23395 A.

### BRIEF SUMMARY

These methods are superior to biological and biochemical substance introduction methods in terms of reproducibility of introduction, a small number of residues (viral vectors and the like) and the like, but are inferior in terms of introduction efficiency or cytotoxicity.

Therefore, an object of the present disclosure is to provide a substance introduction device and a substance introduction method capable of implementing improved introduction efficiency or cytotoxicity using a physical introduction method.

A substance introduction device according to at least one aspect of the present disclosure is a substance introduction device including at least one turbulence grid that generates turbulence in a fluid containing a cell and a substance to be introduced into the cell, in which at least a part of the at least one turbulence grid is formed of a conductive material that applies charge to the cell in the fluid.

As an embodiment of the present disclosure, the substance introduction device is the substance introduction device, in which only a part of the at least one turbulence grid is formed of the conductive material.

As an embodiment of the present disclosure, the substance introduction device is the substance introduction device, in which the charge includes an electric pulse.

As an embodiment of the present disclosure, the substance introduction device is the substance introduction device, in which a flow rate of the fluid is adjustable.

As an embodiment of the present disclosure, the substance introduction device is the substance introduction device, in which the charge is adjustable.

A substance introduction method according to an aspect of the present disclosure is a substance introduction method including a stress/charge applying step of applying, while generating turbulence by flowing a fluid containing a cell and a substance to be introduced into the cell through at least one turbulence grid, charge to the cell in the fluid by a conductive material forming at least a part of the at least one turbulence grid.

As an embodiment of the present disclosure, the substance introduction method is the substance introduction method including a conductive material adjusting step of adjusting a ratio of a portion formed of the conductive material in the at least one turbulence grid.

As an embodiment of the present disclosure, the substance introduction method is the substance introduction method including a flow rate adjusting step of adjusting a flow rate of the fluid.

As an embodiment of the present disclosure, the substance introduction method is the substance introduction method including a charge adjusting step of adjusting the charge.

The present disclosure may provide a substance introduction device and a substance introduction method capable of implementing improved introduction efficiency or cytotoxicity using a physical introduction method.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic diagram illustrating a substance introduction device according to at least one embodiment of the present disclosure;

FIG. 2 is a schematic diagram illustrating a stress/charge application unit of the substance introduction device illustrated in FIG. 1; and

FIG. 3 is a schematic diagram illustrating a state of charge application by a conductive material of the stress/charge application unit illustrated in FIG. 2.

### DETAILED DESCRIPTION

Hereinafter, an embodiment of the present disclosure will be described by way of example in detail with reference to the drawings.

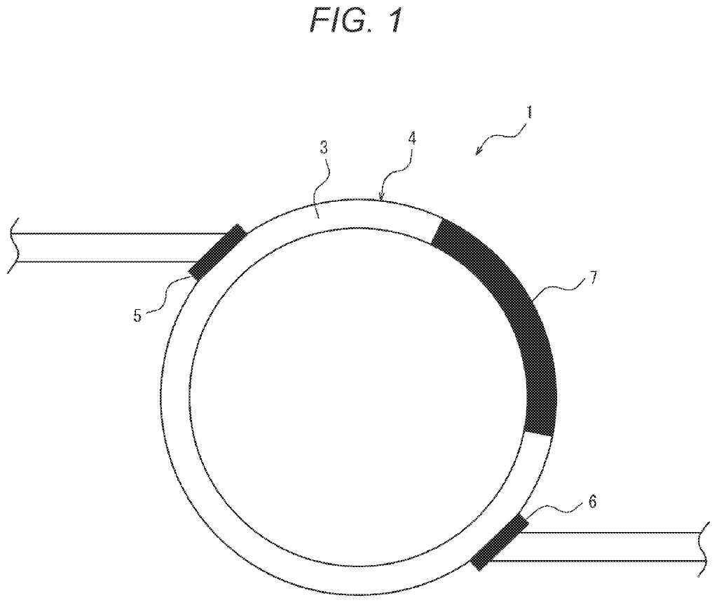

As illustrated in FIGS. 1 to 3, a substance introduction device 1 according to at least one embodiment of the present disclosure is a device that introduces a substance to be introduced to a cell 2 by applying both stress application and charge application as a physical introduction method to the cell 2 in a fluid 3 containing a predetermined amount of cells 2 and the substance to be introduced (that is, the substance that should be introduced into the cell 2) not illustrated.

An amount of cells 2 contained in the fluid 3 is not particularly limited. The cell 2 is not particularly limited, and is, for example, an animal cell, a plant cell, a bacterial cell, and/or the like.

An amount of substances to be introduced contained in the fluid 3 is not particularly limited. The substance to be introduced is not particularly limited, and is, for example, a gene, deoxyribonucleic acid (DNA), ribonucleic acid (RNA), a nucleic acid, a protein, a low molecular compound, a lipid molecule, a liposome, and/or the like.

The fluid 3 is, for example, a suspension obtained by suspending the cell 2 and the substance to be introduced in a buffer.

The substance introduction device 1 includes a closed loop shaped circulation flow path 4, an inlet 5 through which the fluid 3 may be introduced into the circulation flow path 4, and an outlet 6 through which the fluid 3 may be discharged from the circulation flow path 4.

The inlet 5 may have a structure that suppresses, restricts, or prevents backflow of the fluid 3 from the circulation flow path 4. The inlet 5 may have an opening/closing structure that can be opened for introducing the fluid 3 into the circulation flow path 4. The inlet 5 is formed of, for example, a check valve.

The outlet 6 may have a structure that suppresses, restricts, or prevents backflow of the fluid 3 into the circulation flow path 4. The outlet 6 may have an opening/closing structure that can be opened for discharging the fluid 3 from the circulation flow path 4. The outlet 6 is formed of, for example, a check valve.

The circulation flow path 4 includes a stress/charge application unit 7 that applies stress and charge to the cell 2 in the fluid 3.

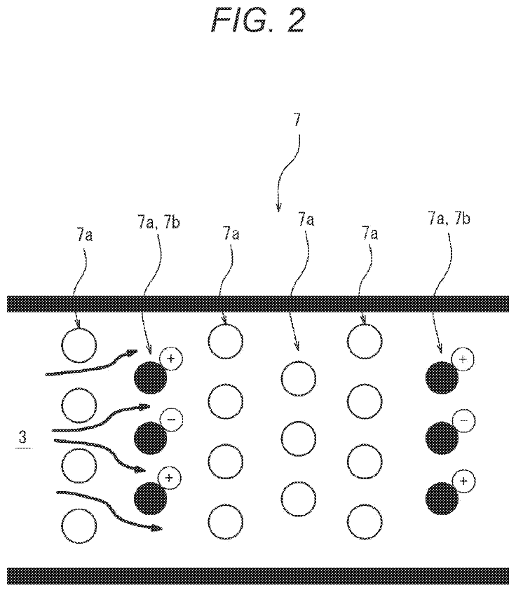

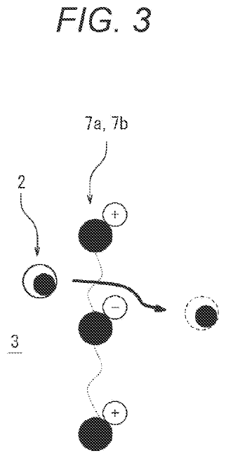

As illustrated in FIG. 2, the stress/charge application unit 7 includes at least one turbulence grid 7a that applies stress such as shear stress to the cell 2 in the fluid 3 by generating turbulence (refer to thick arrows in FIG. 2) in the fluid 3. The turbulence grid 7a is not particularly limited as long as this is a grid capable of generating turbulence, and is, for example, a slit grid, a mesh grid and the like. The slit grid includes, for example, a plurality of bar-shaped members extending in parallel at intervals or only one bar-shaped member. The stress/charge application unit 7 may include a plurality of turbulence grids 7a arranged in a flow direction (e.g., in a direction from the left side to the right side, or vice versa, as illustrated in FIGS. 2 to 3).

The at least one turbulence grid 7a may form a hole, or opening, for introducing the substance to be introduced into the cell 2 on a cell membrane of the cell 2 by applying stress corresponding to a flow rate of the fluid 3 in the stress/charge application unit 7 to the cell 2 in the fluid 3. The flow rate of the fluid 3 may be adjusted in consideration of introduction efficiency and cytotoxicity. The higher the flow rate is, the easier it is to introduce the substance to be introduced into the cell 2, but the more likely it is to cause damage to the cell 2.

At least a part of the at least one turbulence grid 7a is formed of a conductive material 7b that applies charge to the cell 2 in the fluid 3. In some embodiments, only a part of the at least one turbulence grid 7a is formed of the conductive material 7b. The charge may include an electric pulse (e.g., emitted via the conductive material 7b). The conductive material 7b includes, for example, an electrode pair as illustrated in FIGS. 2 to 3 (e.g., arranged side by side and spaced apart from one another by a fluid flow gap). The electrode pair may be provided side by side in a direction orthogonal to the flow direction of the fluid 3 as illustrated in FIGS. 2 to 3, or may be provided side by side in the flow direction of the fluid 3.

The conductive material 7b may form a hole, or opening, for introducing the substance to be introduced into the cell 2 on the cell membrane of the cell 2 according to the charge (e.g., strength, intensity, size, frequency, and/or the like). The charge may be adjusted in consideration of the introduction efficiency and cytotoxicity. The stronger the charge is, the easier it is to introduce the substance to be introduced into the cell 2, but the more likely it is to cause damage to the cell 2.

Therefore, the stress/charge application unit 7 may apply the stress and the charge to the cell 2 in the fluid 3 simultaneously, or at the same time, by applying the charge to the cell 2 in the fluid 3 by the conductive material 7b forming at least a part of the at least one turbulence grid 7a while generating turbulence by flowing the fluid 3 through the at least one turbulence grid 7a.

A ratio of a portion formed of the conductive material 7b in the at least one turbulence grid 7a may be adjusted in consideration of the introduction efficiency and cytotoxicity. The higher the ratio is, the easier it is to introduce the substance to be introduced into the cell 2, but the more likely it is to cause damage to the cell 2.

The stress/charge application unit 7 may be provided over an entire circumference in the circulation flow path 4, or may be provided only in a part of the entire circumference as illustrated in FIG. 1. By providing only in a part of the entire circumference, it is possible to give time to recover disorder occurring in the cell 2 to some extent after the cell 2 is perforated by the stress/charge application unit 7 and before the cell 2 is perforated again by the stress/charge application unit 7, thereby improving the cytotoxicity.

As illustrated in FIG. 1, the circulation flow path 4 includes one stress/charge application unit 7. Note that, the number of stress/charge application units 7 provided in the circulation flow path 4 may be appropriately set. Stated another way, more stress application units 7 may be provided in the circulation flow path than are illustrated in FIG. 1.

The circulation flow path 4 includes a pump (not illustrated) such as a peristaltic pump that circulates the fluid 3 in the circulation flow path 4.

The substance introduction device 1 may introduce the substance to be introduced of an amount corresponding to a degree of circulation (e.g., time, the number of circulations, and/or the like) into the cell 2 by applying the stress and the charge to the cell 2 in the fluid 3 by the stress/charge application unit 7 while circulating the fluid 3 in the circulation flow path 4.

The degree of circulation may be adjusted by adjusting the amount of fluid 3 passing through the inlet 5 and the outlet 6, a timing at which the fluid 3 passes through the inlet 5 and the outlet 6, and/or the like. The fluid 3 containing the cell 2 into which the substance to be introduced is introduced may be discharged from the circulation flow path 4 through the outlet 6.

According to the substance introduction device 1 according to at least one embodiment of the present disclosure, physical perforation methods having different mechanisms of stress application and charge application may be applied simultaneously and to a desired degree in the circulation flow path 4, so that improved introduction efficiency or cytotoxicity may be implemented.

Next, as a substance introduction method according to at least one embodiment of the present disclosure, a substance introduction method using the substance introduction device 1 according to the embodiment described above with reference to FIGS. 1 to 3 will be described by way of example.

The substance introduction method according to at least one embodiment of the present disclosure includes a fluid introducing step, a circulating step, a fluid discharging step, a conductive material adjusting step, a flow rate adjusting step, and a charge adjusting step.

The fluid introducing step is a step of introducing the fluid 3 into the circulation flow path 4 through the inlet 5.

The circulating step is a step of performing a stress/charge applying step while circulating the fluid 3 in the circulation flow path 4 to a desired degree by the above-described pump. The stress/charge applying step is a step of applying the charge to the cell 2 in the fluid 3 by the conductive material 7b forming at least a part of the at least one turbulence grid 7a while generating turbulence by flowing the fluid 3 through the at least one turbulence grid 7a of the stress/charge application unit 7.

The fluid discharging step is a step of discharging the fluid 3 from the circulation flow path 4 through the outlet 6.

The degree of circulation at the circulating step may be adjusted by adjusting the amount of fluid 3 passing through the inlet 5 and the outlet 6, the timing at which the fluid 3 passes through the inlet 5 and the outlet 6 or the like at the fluid introducing step and the fluid discharging step. The fluid 3 containing the cell 2 into which the substance to be introduced is introduced may be taken out of the circulation flow path 4 at the fluid discharging step.

The conductive material adjusting step is a step of adjusting the ratio of the portion formed of the conductive material 7b in the at least one turbulence grid 7a of the stress/charge application unit 7. The ratio may be adjusted in consideration of the introduction efficiency and cytotoxicity as described above. The conductive material adjusting step may be performed simultaneously with the stress/charge applying step (circulating step), or may be performed in advance before the stress/charge applying step (circulating step) is performed.

The flow rate adjusting step is a step of adjusting the flow rate of the fluid 3 in the stress/charge application unit 7 (in the circulation flow path 4). The flow rate of the fluid 3 may be adjusted in consideration of the introduction efficiency and cytotoxicity as described above. The flow rate adjusting step may be performed simultaneously with the stress/charge applying step (circulating step), or may be performed in advance before the stress/charge applying step (circulating step) is performed.

The charge adjusting step is a step of adjusting the charge (e.g., strength, intensity, size, frequency, and/or the like) applied to the cell 2 in the fluid 3 by the stress/charge application unit 7. The charge may be adjusted in consideration of the introduction efficiency and cytotoxicity. The charge adjusting step may be performed simultaneously with the stress/charge applying step (circulating step), or may be performed in advance before the stress/charge applying step (circulating step) is performed.

According to the substance introduction method according to at least some embodiments of the present disclosure, the physical perforation methods having different mechanisms of stress application and charge application may be applied simultaneously and to a desired degree in the circulation flow path 4, so that improved introduction efficiency or cytotoxicity may be implemented.

The above-described embodiment is merely an example of the present disclosure, and various modifications as described below may be made, for example.

The substance introduction device 1 according to at least some embodiments of the present disclosure may be variously modified as long as this is the substance introduction device 1 including the at least one turbulence grid 7a that generates turbulence in the fluid 3 containing the cell 2 and the substance to be introduced into the cell 2 in which at least a part of the at least one turbulence grid 7a is formed of the conductive material 7b that applies the charge to the cell 2 in the fluid 3.

For example, the substance introduction device 1 according to at least one embodiment of the present disclosure may be the substance introduction device 1 including a flow path not in a closed loop shape provided with the stress/charge application unit 7 instead of including the circulation flow path 4.

Note that, the substance introduction device 1 according to at least one embodiment of the present disclosure is, for example, the substance introduction device 1 in which only a part of the at least one turbulence grid 7a is formed of the conductive material 7b.

The substance introduction device 1 according to at least one embodiment of the present disclosure is, for example, the substance introduction device 1 in which the charge includes the electric pulse.

The substance introduction device 1 according to at least one embodiment of the present disclosure is, for example, the substance introduction device 1 in which the flow rate of the fluid 3 may be adjusted.

The substance introduction device 1 according to at least one embodiment of the present disclosure is, for example, the substance introduction device 1 in which the charge may be adjusted.

The substance introduction method according to at least one embodiment of the present disclosure may be variously modified as long as this is the substance introduction method including the stress/charge applying step of applying the charge to the cell 2 in the fluid 3 by the conductive material 7b forming at least a part of the at least one turbulence grid 7a while generating turbulence by flowing the fluid 3 containing the cell 2 and the substance to be introduced into the cell 2 through the at least one turbulence grid 7a.

For example, the substance introduction method according to at least one embodiment of the present disclosure may be the substance introduction method of performing the stress/charge applying step while flowing the fluid 3 into the flow path not in the closed loop shape instead of including the circulating step.

The substance introduction method according to at least one embodiment of the present disclosure is, for example, the substance introduction method including the conductive material adjusting step of adjusting the ratio of the portion formed of the conductive material 7b in the at least one turbulence grid 7a.

The substance introduction method according to at least one embodiment of the present disclosure is, for example, the substance introduction method including the flow rate adjusting step of adjusting the flow rate of the fluid 3.

The substance introduction method according to at least one embodiment of the present disclosure is, for example, the substance introduction method including the charge adjusting step of adjusting the charge.

The exemplary systems and methods of this disclosure have been described in relation to a substance introduction device and method. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A substance introduction device comprising:
at least one turbulence grid that generates turbulence in a fluid containing a cell and a substance to be introduced into the cell, wherein
at least a part of the at least one turbulence grid is formed of a conductive material that applies charge to the cell in the fluid.

2. The substance introduction device according to claim 1, wherein only a part of the at least one turbulence grid is formed of the conductive material.

3. The substance introduction device according to claim 1, wherein the charge includes an electric pulse.

4. The substance introduction device according to claim 1, wherein a flow rate of the fluid is adjustable.

5. The substance introduction device according to claim 1, wherein the charge is adjustable.

6. The substance introduction device according to claim 1, further comprising:
a circulation flow path configured to receive and convey the fluid through the at least one turbulence grid.

7. The substance introduction device according to claim 6, further comprising:
an inlet interconnected with the circulation flow path and disposed on a first side of the at least one turbulence grid, wherein the inlet is configured to receive the fluid; and
an outlet interconnected with the circulation flow path on a second side of the at least one turbulence grid, wherein the outlet is configured to discharge the cell comprising the substance to be introduced into the cell from the circulation flow path.

8. The substance introduction device according to claim 7, wherein the at least one turbulence grid comprises a plurality of bar-shaped members arranged adjacent one another in the circulation flow path, and wherein the plurality of bar-shaped members are configured to apply stress to the cell as the fluid moves through the at least one turbulence grid.

9. The substance introduction device according to claim 7, wherein the circulation flow path is arranged in a closed loop shape.

10. A substance introduction method comprising: a stress/charge applying step of applying, while generating turbulence by flowing a fluid containing a cell and a substance to be introduced into the cell through at least one turbulence grid, charge to the cell in the fluid by a conductive material forming at least a part of the at least one turbulence grid.

11. The substance introduction method according to claim 10, further comprising: a conductive material adjusting step of adjusting a ratio of a portion formed of the conductive material in the at least one turbulence grid.

12. The substance introduction method according to claim 10, further comprising: a flow rate adjusting step of adjusting a flow rate of the fluid.

13. The substance introduction method according to claim 10, further comprising: a charge adjusting step of adjusting the charge.

14. A substance introduction method comprising:
introducing a fluid comprising a cell and a substance into a circulation flow path;
flowing the fluid through at least one turbulence grid arranged in the circulation flow path, wherein flowing the fluid through the at least one turbulence grid generates turbulence in the fluid; and
applying a charge to the fluid simultaneously as the fluid passes through the at least one turbulence grid and as the turbulence in the fluid is generated, causing the substance to be introduced into the cell.

15. The substance introduction method of claim 14, wherein at least one of the charge and the turbulence in the fluid forms an opening in a membrane of the cell, and wherein the substance is introduced into the cell via the opening.

16. The substance introduction method of claim 15, wherein at least a part of the at least one turbulence grid comprises a conductive material, and wherein applying the charge comprises emitting an electric pulse via the conductive material.

17. The substance introduction method of claim 16, wherein the conductive material is an electrode pair arranged side by side with a fluid flow gap disposed therebetween.

18. The substance introduction method of claim 14, further comprising:
adjusting a flow rate of the fluid passing through at least one turbulence grid based on an introduction efficiency and cytotoxicity for the cell.

19. The substance introduction method of claim 18, further comprising:
adjusting a charge intensity of the charge based on the introduction efficiency and cytotoxicity for the cell.

20. The substance introduction method of claim 14, wherein the circulation flow path is arranged in a closed loop shape, wherein the fluid is introduced into the circulation flow path via an inlet in fluid communication with the circulation flow path, and wherein the fluid is discharged from the circulation flow path via an outlet in fluid communication with the circulation flow path after the substance is introduced into the cell.
